(19) United States
(12) Patent Application Publication
(10) Pub. No.: US 2005/0244512 A1
(43) Pub. Date: Nov. 3, 2005

(54) Ophthalmic fluid and method of delivering same

(76) Inventors: Nancy Melberg Holekamp, St. Louis, MO (US); David Cy Beebe, St. Louis, MO (US); Ying-Bo Shui, St. Louis, MO (US); Steven Bassnett, Chesterfield, MO (US); Richard McNulty, New South Wales (AU)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/836,569

(22) Filed: May 1, 2004

(51) Int. Cl. A61K 38/05; A61K 33/14

(52) U.S. Cl. 424/678; 514/18; 424/680

(57) ABSTRACT

A method for preparing and infusing an ophthalmic fluid into an eye includes de-oxygenating the fluid and infusing the fluid into the eye. In another aspect, the fluid is warmed such that it enters the eye within a temperature range of about 30 degrees C. to about 37 degrees C.

### STATEMENT REGARDING FEDERALLY SPONSORED RESEARCH & DEVELOPMENT

This research was partially funded by US NIH grant EYO4853. The Government may have certain rights in the invention.

### BACKGROUND OF THE INVENTION

This invention relates generally to medical procedures involving the eye and, more particularly, to ophthalmic fluids used in eye surgeries and methods for supplying those fluids to an eye.

The vitreous is a transparent gel that fills at least a portion of the eye, typically from the iris to the retina. The vitreous helps the eye hold its shape. The vitreous is susceptible to several afflictions that impair vision by damaging its transparency. Some of these afflictions are infections, injuries, bleeding, and blood vessels growing into the vitreous. In addition, on occasion the retina will fall into the vitreous, a condition called retinal detachment. For any of these above listed afflictions, it may be necessary to surgically remove the vitreous and substitute a suitable solution during the surgery. The removal and substitution procedure is called vitrectomy. The solution is added to the eye at a sufficient rate such that the eye maintains its pressure and shape during and after the procedure.

During a typical vitrectomy procedure three probes (a vitrector, a light source, and an infusion canula) are inserted into the eye through small incisions in the sclera. The vitreous humor is removed using the vitrector, a miniature cutting and aspiration tool. The infusion line serves to keep the pressure constant in the eye during the procedure. As the vitreous is aspirated, an infusion fluid is provided to the eye with a slight positive pressure through the infusion canula. During the operation (which may take several hours), a total of approximately 400 ml of solution is infused into the eye and aspirated along with the vitreous. The infusion fluid is typically a physiologic saline solution similar in properties to the material being removed from the eye. A notable exception is that the solution is oxygenated (partially equilibrated with room air) and is supplied to the eye at a temperature commensurate with the room temperature of the operating room, i.e., around 24 degrees C. to about 27 degrees C.

Although vitrectomy is an effective treatment, it is associated with an extremely high incidence of post-surgical cataracts. For example, within six months of vitrectomy, approximately 21% of patients develop cataract and within 12 months of the vitrectomy, this number rises to approximately 63%. The most common type of cataract seen after vitrectomy is nuclear cataract. Eventually, cataract surgery is required in a majority of the cases.

### BRIEF DESCRIPTION OF THE INVENTION

In one aspect, a deoxygenated ophthalmic fluid is provided that may include at least one reducing agent.

In another aspect, a method for preparing and infusing an ophthalmic fluid into the eye is provided. The method includes de-oxygenating the fluid and infusing the fluid into the eye.

In another aspect, a method for preparing and infusing an ophthalmic fluid into an eye is provided. The method includes warming the fluid to within a range of about 30 degrees C. to about 37 degrees C.

In another aspect, a method for performing a vitrectomy is provided. The method includes obtaining a de-oxygenated infusion fluid, removing the vitreous of an eye, and infusing the infusion fluid into the eye.

### BRIEF DESCRIPTION OF THE DRAWINGS

FIG. 1 is a set of graphs depicting the effect of temperature on pO₂ in the nucleus of a human donor lens.

### DETAILED DESCRIPTION OF THE INVENTION

Exemplary embodiments of compositions of fluids and methods of supplying the fluids are described below. In one embodiment, the fluid includes reduced glutathione and the method includes de-oxygenating the fluid and adding reduced glutathione to the fluid. The fluid is supplied to the eye through an oxygen impermeable tube within a temperature range of about 30 degrees C. to about 37 degrees C. to the eye. Although exemplary embodiments are described herein, the fluid and methods of delivery are not limited to those specific embodiments.

A factor causing damage to an eye during surgery is the traumatic change in environment. Exposure to the atmosphere or to a foreign wetting solution presents a possibly hostile environment for the cells in the eye. For example, exposure to a particular fluid may present a challenge for the cells involved. Accordingly, solutions are provided to the eye during a vitrectomy that attempt to approximate the natural bodily fluids in the eye.

It is believed that fluid delivered to the eye at conditions that mimic the conditions in the eye, will reduce the occurrence of post-vitrectomy nuclear cataracts. Such conditions include a temperature at or near body temperature and an oxygen level at or near the oxygen level of the vitreous body gel that is being replaced.

Due to experiments recently conducted on cadaveric lenses, it is believed that the lens core (the center tissue in the lens) is flooded with oxygen during a typical vitrectomy, due, at least in part, to the cooling of the lens in the presence of oxygenated solutions. It is believed that the introduction of high concentrations of oxygen into the previously hypoxic core of the lens is a direct cause of post-operative cataracts. Two aspects of the fluid are believed to play a role in providing the high concentration of oxygen to the lens. The first aspect is that fluid is typically provided to the lens in an oxygenated state (partially equilibrated with room air). The second aspect is that the fluid is delivered to the eye at room temperature (typically between about 24 degrees C. and 27 degrees C.). The cool temperature of the fluid causes the lens to cool which allows more oxygen to enter the lens.

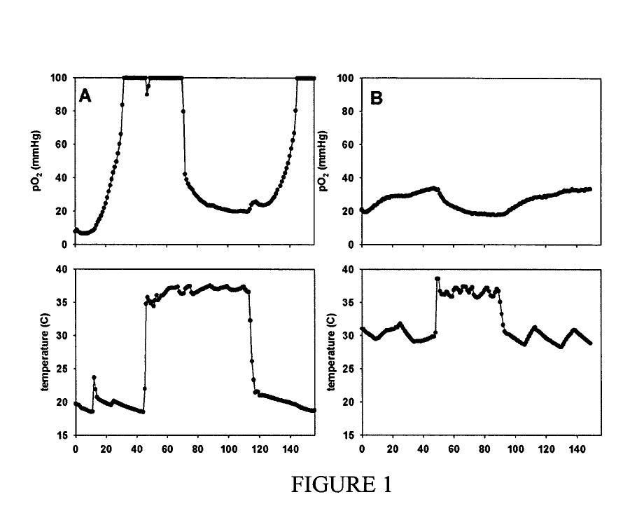

FIG. 1 illustrates the effect of temperature on oxygen content (pO₂) in the nucleus of human donor lenses. Isolated human lenses were incubated in solutions equilibrated with atmospheric oxygen (about 150 mmHg or 21% O₂). In A, the pO₂ and temperature were monitored in the lens center as the temperature of the bathing solution was alternated between 37 degrees C. and 20 degrees C. At 37 degrees C., pO₂ in the lens core is less than 10 mmHg. At room temperature, the pO₂ at the lens core increases to greater than 100 mmHg. The effect appears to be reversible. In B, the lens core is maintained in a relatively hypoxic condition (less than 35 mmHg) provided the tissue temperature does not fall below 30 degrees C.

In accordance with one embodiment of the present invention, an ophthalmic fluid is prepared for performing a surgery on an eye. The ophthalmic fluid is de-oxygenated and then stored in an oxygen impermeable storage bottle. The process of de-oxygenating the fluid includes introducing nitrogen gas into the fluid so that the nitrogen replaces most of the oxygen. The nitrogen gas is delivered from a tank of compressed pure nitrogen and is passed through a 0.45 micrometer filter to prevent contaminants from entering the solution. The nitrogen gas enters the bottle containing the fluid through a port in the bottom of the bottle. The nitrogen gas is bubbled through the fluid and exits through an opening at the top of the bottle. Nitrogen is bubbled for approximately 10 minutes to remove the majority of the oxygen. In one embodiment, nitrogen is bubbled through the fluid until the oxygen partial pressure is less than about 10 mmHg. In another embodiment, nitrogen is bubbled through the fluid until the oxygen partial pressure is between about 10 mmHg and about 2 mmHg. More particularly, nitrogen is bubbled through the fluid until the oxygen partial pressure is about 5 mmHg or less (approximately 0.5% oxygen).

In an exemplary embodiment, 500 cc of sterile ophthalmic fluid is contained in an oxygen impermeable bottle, e.g., glass, which is capped with an oxygen impermeable rubber stopper. A sterile needle of approximately 18 gauge bore and 6 inches long is used to penetrate the rubber stopper so the needle rests within the fluid. The end of the needle outside the bottle is attached to tubing connected to a tank of 100% nitrogen gas. Under low pressure nitrogen gas is bubbled into the ophthalmic fluid for at least 10 minutes. Also inserted through the rubber stopper alongside the needle is a one-way valve for the egress of excess oxygen or nitrogen gases during the bubbling procedure. The bubbling of nitrogen gas in the ophthalmic fluid as described above will result in a fluid oxygen tension of 2 to 10 mm Hg pressure, thus effectively de-oxygenating the fluid.

In one embodiment, the fluid is de-oxygenated in the same location at which it is formed. For example, after the solution has been created, it is immediately de-oxygenated and then stored in an oxygen impermeable storage bottle. Alternatively, the fluid is de-oxygenated at the location at which the procedure is to be performed.

A first exemplary ophthalmic fluid is set forth below in Table 1.

TABLE 1

| Component | Amount (Wt %) |
|---|---|
| sodium chloride | 0.64% |
| potassium chloride | 0.075% |
| calcium chloride dihydrate | 0.048% |
| magnesium chloride | 0.03% |
| sodium acetate | 0.39% |
| sodium citrate dihydrate | 0.17% |
| sodium hydroxide | to adjust pH in water |
| or hydrochloric acid. | |

A fluid having the composition as listed in Table 1 is currently available from Alcon Laboratories, Inc., Fort Worth, Tex. 76134. A second exemplary ophthalmic fluid is set forth below in Table 2.

TABLE 2

| Component | Amount (mg/ml) |
|---|---|
| sodium chloride | 07.14 mg |
| potassium chloride | 0.38 mg |
| calcium chloride dihydrate | 0.154 mg |
| magnesium chloride | 0.20 mg |
| dibasic sodium phosphate | 0.42 mg |
| sodium bicarbonate | 2.10 mg |
| dextrose | 0.92 mg |
| glutathione | 0.184 mg |
| sodium hydroxide | to adjust pH in water |
| or hydrochloric acid. | |

A fluid having the composition as listed in Table 2 is currently available from Alcon Laboratories, Inc., Fort Worth, Tex. 76134.

In one embodiment, after the ophthalmic fluid has been deoxygenated, at least one reducing agent is added to the fluid. An exemplary reducing agent is reduced glutathione. The glutathione is added to the fluid by injecting the glutathione into the storage bottle containing the deoxygenated fluid in an amount that provides the desired final concentration of reduced glutathione. Alternatively, the reduced glutathione is combined with the deoxygenated fluid during infusion with a proportioning pump that adds an appropriate amount of reduced glutathione to the fluid during infusion. In a further embodiment, the glutathione is added to the fluid by other means that result in the desired final concentration of glutathione in the fluid entering the eye. For example, 10 Mm sterile reduced glutathione is dissolved in the infusion fluid prior to infusing the fluid into the eye.

Although reduced glutathione has been described as a reducing agent in the exemplary embodiment, it should be understood that other reducing agents can also be used. Other exemplary reducing agents that can be used in the present invention include: ascorbic acid (vitamin C), N-acetyl cysteine, D- or L-cysteine, D- or L-methionine, dithiothreitol, dithioerythritol, and mercaptoethanol.

The method for providing de-oxygenated fluid to the eye includes de-oxygenating the fluid as described above. A reducing agent is then added to the deoxygenated fluid. The fluid is then delivered to the eye through an oxygen-impermeable tubing during the procedure under a positive pressure. The positive pressure is controlled, in one embodiment, with a pneumatic pump within the vitrectomy machine. The gas chosen to power the pneumatic pump is nitrogen since the gas from the pump may contact the fluid.

Another aspect of the invention is that the fluid is warmed prior to infusion into the eye. The temperature of the warmed fluid as it enters the eye is between about 30 degrees C. and about 37 degrees C. More particularly, the temperature of the warmed fluid as it enters the eye is between about 32 degrees C. and about 35 degrees C. More particularly still, the temperature of the warmed fluid as it enters the eye is about 34 degrees C.

In an exemplary embodiment, the fluid is warmed by a fluid warming device. An exemplary device is WarmFlo, model FW-538 and WF-100 (Mallinckrodt, Inc., St. Louis, Mo.). The fluid warmer utilizes a sterile, disposable, heat exchange cassette. Similar devices are used to warm blood and I.V. solutions to help maintain normal body temperature of patients undergoing surgical procedures. In use, the infusion fluid is passed through the fluid warmer, which is positioned relatively close to the patient's head. The positioning helps to alleviate cooling of the fluid after it has been warmed. In one embodiment, the fluid warmer is equipped with an audible over-temperature alarm. If the alarm sounds, then the fluid flow to the eye is quickly turned off.

The method of providing warmed infusion fluid to the eye includes attaching the fluid warmer to a drip stand and placing the warmer near the head of the patient. The temperature for the warmed fluid is set to between about 30 degrees C. and about 37 degrees C. In one aspect, the temperature for the warmed fluid is set to between about 32 degrees C. and about 35 degrees C. In a further aspect, the temperature for the warmed fluid is set to about 34 degrees C. The irrigation solution bottle is attached to the sterile irrigation tubing as in a normal vitrectomy. The irrigation tubing is attached to the proximal end of the sterile heat exchange unit. The distal end of the heat exchange unit is attached to a standard 3-way tap and silicon tubing going to the eye. The irrigation tubing and heat exchange unit is primed with irrigation solution. The vitrectomy is then conducted. This warming method can be practiced with a de-oxygenated fluid by replacing the silicon tubing with an oxygen impermeable tubing, thus keeping oxygen out of the fluid during its transit from the storage bottle to the eye.

While the invention has been described in terms of various specific embodiments, those skilled in the art will recognize that the invention can be practiced with modification within the spirit and scope of the claims.

1. A deoxygenated ophthalmic fluid comprising at least one reducing agent.

2. A fluid in accordance with claim 1 wherein said reducing agent is glutathione.

3. A fluid in accordance with claim 1 wherein said fluid has a temperature of about 30 degrees C. to about 37 degrees C.

4. A fluid in accordance with claim 1 wherein said fluid has a temperature of about 32 degrees C. to about 35 degrees C.

5. A fluid in accordance with claim 1 wherein said fluid has a temperature of about 34 degrees C.

6. A fluid in accordance with claim 1 further comprising:
sodium chloride;
potassium chloride;
calcium chloride dihydrate;
magnesium chloride;
sodium acetate;
sodium citrate dihydrate; and
at least one of sodium hydroxide and hydrochloric acid.

7. A fluid in accordance with claim 1 further comprising:
sodium chloride;
potassium chloride;
calcium chloride dihydrate;
magnesium chloride;
dibasic sodium phosphate;
sodium bicarbonate;
dextrose; and
at least one of sodium hydroxide and hydrochloric acid.

8. A method for preparing and infusing an ophthalmic fluid into the eye, said method comprising:
de-oxygenating the fluid; and
infusing the fluid into the eye.

9. A method in accordance with claim 8 further comprising:
maintaining the de-oxygenated fluid in an oxygen impermeable container; and
transporting the de-oxygenated fluid from the oxygen impermeable container to the eye through an oxygen impermeable tube such that the oxygen partial pressure of the fluid is less than about 10 mmHg.

10. A method in accordance with claim 8 further comprising warming the fluid to within a range of about 30 degrees C. to about 37 degrees C.

11. A method in accordance with claim 8 wherein the fluid is warmed such that it enters the eye within a range of about 30 degrees C. to about 37 degrees C.

12. A method in accordance with claim 8 wherein the fluid is warmed such that it enters the eye within a range of about 32 degrees C. to about 35 degrees C.

13. A method in accordance with claim 8 wherein the fluid is warmed such that it enters the eye at about 34 degrees C.

14. A method in accordance with claim 8 wherein said fluid is de-oxygenated by bubbling nitrogen through the fluid.

15. A method in accordance with claim 8 further comprising adding a reducing agent to the de-oxygenated fluid.

16. A method in accordance with claim 8 further comprising adding reduced glutathione to the de-oxygenated fluid.

17. A method for preparing and infusing an ophthalmic fluid into an eye, said method comprising warming the fluid to within a range of about 30 degrees C. to about 37 degrees C.

18. A method in accordance with claim 17 comprising warming the fluid to within a range of about 32 degrees C. to about 35 degrees C.

19. A method in accordance with claim 17 comprising warming the fluid to about 34 degrees C.

20. A method in accordance with claim 17 wherein the fluid is warmed such that it enters the eye within a range of about 30 degrees C. to about 37 degrees C.

21. A method in accordance with claim 17 wherein the fluid is warmed such that it enters the eye within a range of about 32 degrees C. to about 35 degrees C.

22. A method in accordance with claim 17 wherein the fluid is warmed such that it enters the eye at about 34 degrees C.

23. A method in accordance with claim 17 further comprising adding reduced glutathione to the fluid.

24. A method in accordance with claim 17 further comprising:
de-oxygenating the fluid; and
adding a reducing agent to the de-oxygenated fluid.

25. A method in accordance with claim 17 wherein the reducing agent is reduced glutathione.

26. A method in accordance with claim 17 further comprising delivering the fluid to the eye with a positive pressure.

27. A method for performing a vitrectomy comprises:
obtaining a de-oxygenated infusion fluid;
removing the vitreous of an eye; and
infusing the infusion fluid into the eye.

28. A method in accordance with claim 27 further comprising adding a reducing agent to the de-oxygenated fluid.

29. A method in accordance with claim 27 further comprising adding reduced glutathione to the de-oxygenated fluid.

30. A method in accordance with claim 27 further comprising warming the fluid to a temperature of between about 30 degrees C. to about 37 degrees C.

31. A method in accordance with claim 27 further comprising infusing the infusion fluid to the eye such that the fluid enters the eye between a temperature range of about 30 degrees C. to about 37 degrees C.

32. A method in accordance with claim 27 further comprising infusing the infusion fluid to the eye such that the fluid enters the eye between a temperature range of about 32 degrees C. to about 35 degrees C.

33. A method in accordance with claim 27 further comprising infusing the infusion fluid to the eye such that the fluid enters the eye at about 34 degrees C.

34. A method in accordance with claim 27 further comprising warming the fluid to a temperature of between about 30 degrees C. to about 37 degrees C.

35. A method in accordance with claim 27 further comprising warming the fluid to a temperature of between about 32 degrees C. to about 35 degrees C.

36. A method in accordance with claim 27 further comprising warming the fluid to a temperature of about 34 degrees C.

37. A method in accordance with claim 27 wherein said step of obtaining de-oxygenated infusion fluid comprises bubbling nitrogen through oxygenated infusion fluid to de-oxygenate the fluid.
